(19) United States
(12) Patent Application Publication
(10) Pub. No.: US 2019/0318802 A1
(43) Pub. Date: Oct. 17, 2019

(54) METHOD AND APPARATUS FOR IMPROVED DETERMINATION OF NODE INFLUENCE IN A NETWORK

(71) Applicant: UNIVERSITY OF FLORIDA RESEARCH FOUNDATION, INCORPORATED, Gainesville, FL (US)

(72) Inventors: David TRAN, Gainesville, FL (US); Son Bang LE, Gainesville, FL (US)

(21) Appl. No.: 16/340,738

(22) PCT Filed: Oct. 13, 2017
(86) PCT No.: PCT/IB17/56376

§ 371 Date: Apr. 10, 2019

Related U.S. Application Data
(60) Provisional application No. 62/408,045, filed on Oct. 13, 2016.

(51) Int. Cl. G16B 25/00; G16B 5/00

(52) U.S. Cl. CPC G16B 25/00; G16B 5/00

(57) ABSTRACT

Example embodiments of the present invention address problems in computational biology and network theory. As mentioned above, example embodiments enable the determination of where to set a threshold cutoff level to maximize sensitivity of a gene expression profile while minimizing the false discovery rate (FDR). Some example embodiments exploit the filtered gene expression profile to discover potential master regulators that may be targetable by various perturbagen-based treatments. Other example embodiments facilitate the harvesting of meaningful data from other type of network and node statistics inputs, even those not related specifically to computational biology or the exploitation of inferred gene expression profiles.

### CROSS-REFERENCE TO RELATED APPLICATIONS

This application claims priority to U.S. Provisional Patent Application No. 62/408,045, filed Oct. 13, 2016, the entire contents of which are incorporated herein by reference.

### STATEMENT REGARDING FEDERALLY SPONSORED RESEARCH OR DEVELOPMENT

This invention was made with government support under K08 CA160824 awarded by the National Institutes of Health. The government has certain rights in the invention.

### BACKGROUND

Recent genomics studies suggest that cancers originate from a founding stem-like cell clone that emerges after sustaining a series of initiating and cooperative alterations that are passed on such that all subclones contain the founding alterations (i.e. the core common master regulators) and hence are targetable. An example of this clonal evolution is shown in FIG. 1, which shows an example of how founding mutations (rectangles) are thought to be passed on to subclones. As the number of potential founding alterations is surprisingly small (i.e. 8-12), many founding alterations are expected to be common across different tumors of the same type or even of different types. Therefore to achieve therapeutic success, core master regulators specific only to the founding stem-like cells must first be systemically identified across multiple cancerous tumors and functionally validated, followed by simultaneous targeting of these core factors to achieve maximal efficacy with minimal toxicity.

Founding alterations may produce “imprints” on the global gene regulatory network that may persist as the founding clone morphs into subclones and may be traceable across subclones. However, to understand the biological implications of these genomic alterations requires novel analytic tools to interrogate large-scale gene expression profiles to provide information on cancer cells' behaviors caused by interactions between the founding alterations and the tumor microenvironment. Gene expression profiles can then be used to infer the global and local networks that control such behaviors. This can be achieved using reverse engineering tools designed to scale up to the complexity of mammalian cells by applying a theoretical information approach to infer gene networks using gene expression data.

However, network inference algorithms using gene expression profiling require the use of many simultaneous statistical inferences, which greatly increases the potential for the appearance of “false discoveries” (the appearance of relationships between genes that do not actually exist). Accordingly, gene expression profiling techniques suffer insofar as it is difficult to differentiate the meaningful genetic relationships from the false discoveries. Thus, there is a need in the art for methods, apparatuses, systems, computing devices, and/or the like for determining where to set a threshold cutoff level to maximize sensitivity of a gene expression profile-based network inference algorithm, while minimizing the false discovery rate (FDR).

Moreover, to discover targetable master regulators within a large gene network requires the ability to rank various genes (or nodes) in the network. While node importance scoring methodologies exist, an improved scoring framework would improve the ability of researchers in identifying potential master regulators relating to specific biological processes, both normal and pathologic, including cancers. Separately, an improved node importance scoring framework provides benefits as a tool for harvesting meaningful data from any type of network and node statistics inputs, even those not related specifically to gene expression profiles. Thus, there is a need in the art for methods, apparatuses, systems, computing devices, and/or the like that can improve upon existing node importance scoring systems.

### BRIEF SUMMARY

Some embodiments of the present invention provide methods, apparatus, systems, computing devices, computing entities, and/or the like, for discovering targetable master regulators within a large gene network. Some embodiments of the present invention provide methods, apparatus, systems, computing devices, computing entities, and/or the like, for improving upon existing node importance scoring systems more generally.

For instance, a method is provided herein for utilizing a computational pipeline to generate a highly reliable regulatory network from gene expression data. The method includes receiving, by an analysis computing entity, an initial set of gene expression values, and generating, by the analysis computing entity, a real dataset and a number of randomized datasets from the initial set of gene expression values. The method further includes applying, by the analysis computing entity, a bootstrap procedure to the real dataset and the randomized datasets to create a series of bootstrap files corresponding to the datasets. For each of the datasets, the method includes processing, by the analysis computing entity, the corresponding series of bootstrap files to generate a set of bootstrap adjacency matrix files, wherein each adjacency matrix file includes an entry for each hub-gene contained in the corresponding bootstrap file, wherein the entry for each hub-gene identifies corresponding edges, and wherein each edge comprises a connection for the hub-gene along with mutual information corresponding to the connection. The method further includes performing, by the analysis computing entity, a consensus procedure that utilizes each set of bootstrap adjacency matrix files to generate a single consensus adjacency matrix file for the corresponding dataset, wherein each consensus adjacency matrix file identifies only a subset of the edges that occur in the set of bootstrap adjacency matrix files. The method further includes determining, by the analysis computing entity and based on the generated consensus adjacency matrix files, significance thresholds for the set of gene expression values. And the method further includes filtering, by the analysis computing entity, the consensus adjacency matrix file for the real dataset using the determined significance thresholds to produce a gene expression network stripped of low-significance edges.

In some embodiments, the method further includes receiving, by the analysis computing entity, a user-specified number of bootstrap rounds and sample size, wherein the series of bootstrap files created in the bootstrap procedure are created based on the number of bootstrap rounds and the sample size.

In some embodiments, processing the series of bootstrap files to generate the set of bootstrap adjacency matrix files utilizes a reverse engineering tool for reconstruction of cellular networks.

In some embodiments, the method further includes determining the subset of the edges that occur in a particular set of bootstrap adjacency matrix files to identify in the consensus adjacency matrix for a corresponding dataset. In some such embodiments, this determination is performed by calculating, by the analysis computing entity, a support level for each edge, calculating, by the analysis computing entity, a false-positive rate (FPR) for each edge, and selecting, by the analysis computing entity, only those edges having a support level and FPR above a predetermined value.

In some embodiments, performing the consensus procedure further includes generating, by the analysis computing entity, a counts file for each consensus adjacency matrix, wherein the counts file identifies a support level of each edge in the consensus adjacency matrix, and generating, by the analysis computing entity, a statistics file that records, for each edge in the consensus adjacency matrix, the support level of the edge, the FPR of the edge, and a sum of the mutual information of the edge, as taken from the bootstrap adjacency matrix files, wherein the significance thresholds for the set of gene expression values are based on the counts file and the statistics file.

Other embodiments are contemplated as well. For instance, another method is provided for calculating importance scores for nodes in a network. The method includes steps of (a) receiving, by the analysis computing entity, an initial dataset describing a network, (b) extracting, by the analysis computing entity, one or more subnetworks from the initial dataset, (c) calculating, by the analysis computing entity, individual scores for each node in the one or more subnetworks, (d) calculating, by the analysis computing entity, neighborhood scores for each node in the one or more subnetworks, (e) generating, by the analysis computing entity, a combined node score for each node in the one or more subnetworks, and (f) iteratively refining, by the analysis computing entity, the combined node scores.

In some embodiments, calculating an individual score (indscore) for a given node i in the network comprises applying the formula:

indscoreᵢ=Πₖ₌₁ⁱstatᵢ^{k},

where statᵢ^{k} is a k-th statistic selected from a list of gene statistics.

In some embodiments, calculating a neighborhood score (nbhscore) for a given node i in the network comprises applying the formula:

$${nbhscore}_{i} = {\sum }_{s = 1}^{step} ( \frac{1}{{s}^{o}} * {\sum }_{j = 1}^{{n}_{s}} ( {w}_{i , j}^{p} * {indscore}_{j} ) ) ,$$

where step represents a number of steps from node i to neighborhood nodes;

$$\frac{1}{{s}^{o}}$$

comprises a weight penalty based on how far a given node s is from node i; w_{i,j}^{p} is a weight of closeness between nodes i and j; and nₛ is a number of neighbors of node i that require s steps to reach node i.

In some embodiments, calculating a neighborhood score (nbhscore) for a given node i in the network comprises applying the formula:

$${nbhscore}_{i} = {\sum }_{s = 1}^{step} ( \frac{1}{{s}^{o}} * ( \frac{1}{{n}_{s}} ) * {\sum }_{j = 1}^{{n}_{s}} ( {w}_{i , j}^{p} * {indscore}_{j} ) ) ,$$

where step represents a number of steps from node i to neighborhood nodes;

$$\frac{1}{{s}^{o}}$$

comprises a weight penalty based on how far a given node s is from node i; w_{i,j}^{p} is a weight of closeness between nodes i and j; and nₛ is a number of neighbors of node i that require s steps to reach node i.

In some embodiments, generating the combined node score for a given node in the network includes one of: setting the combined node score equal to the individual score for the node; setting the combined node score equal to the neighborhood score for the node; setting the combined node score equal to the neighborhood score for the node plus a product produced by multiplying the individual score for the node by a value comprising a number of other nodes in the neighborhood of the node; or setting the combined node score equal to the individual score for the node multiplied by the neighborhood score for the node.

And in some embodiments, iteratively refining the combined node scores includes repeating steps (b), (c), (d), and (e) a predetermined number of time or until convergence is reached.

Still other embodiments are contemplated. For instance, another method is provided for identifying and validating one or more master regulators and biomarkers. The method includes generating, by an analysis computing entity, a gene expression network using, as described herein. The method further includes calculating, by the analysis computing entity, importance scores for the genes in the gene expression network, using steps as described herein. The method then includes identifying a predetermined number of genes having the highest calculated importance scores, and selecting a set of core master regulators based on the predetermined number of genes having the highest calculated importance scores. In some embodiments, the method may further include testing candidate perturbagens to identify a best combination of perturbagens based on the selected set of core master regulators. In still further embodiments, the method may also include developing a predictive test to forecast a response to the best combination of perturbagens.

While described herein using example method steps, it will be understood that the present invention also contemplates apparatuses and computer program products for the above-noted purposes. For instance, an apparatus are provided herein that includes at least one processor and at least one memory storing computer program code. In various embodiments, the at least one memory and the computer program code are configured to, with the processor, cause the apparatus to perform the various combinations of steps recited above. And a computer program product is provided comprising at least one non-transitory computer-readable storage medium. In various embodiments, the at least one non-transitory computer-readable storage medium has computer-executable program code instructions stored therein, the computer-executable program code instructions comprising program code instructions that, when executed, cause a computer to perform the various combinations of steps recited above.

The above summary is provided merely for purposes of summarizing some example embodiments to provide a basic understanding of some aspects of the invention. Accordingly, it will be appreciated that the above-described embodiments are merely examples and should not be construed to narrow the scope or spirit of the invention in any way. It will be appreciated that the scope of the invention encompasses many potential embodiments in addition to those here summarized, some of which will be further described below.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is an example diagram of the clonal evolution of a cancerous cell.

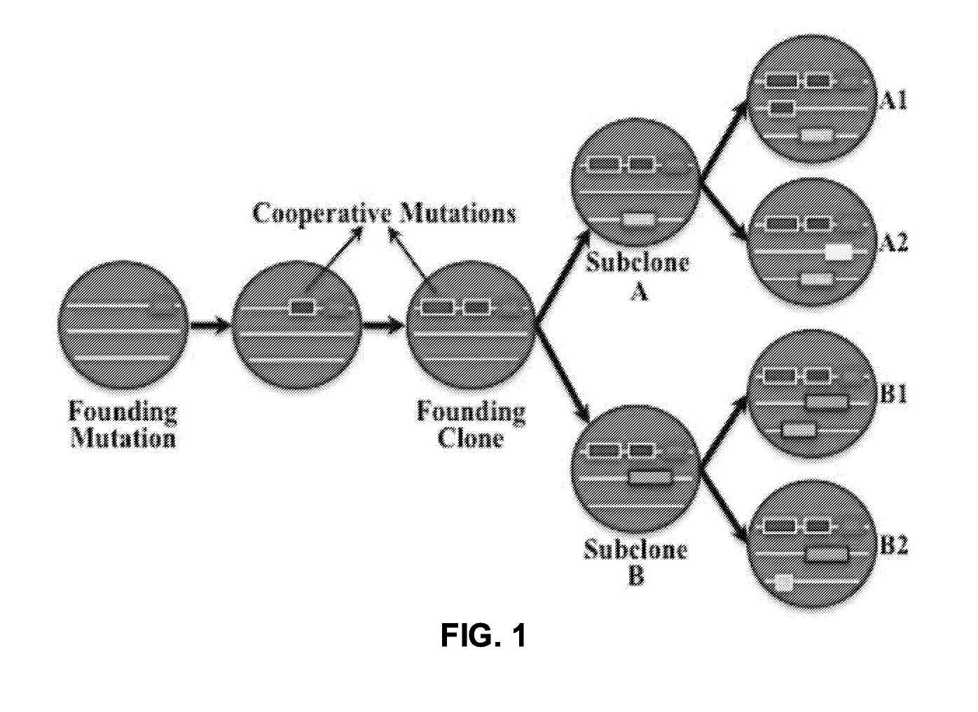

FIG. 2 is an overview of a system that can be used to practice embodiments of the present invention.

FIG. 3 is an exemplary schematic diagram of an analysis computing entity according to one embodiment of the present invention.

FIG. 4 provides a flowchart illustrating operations and processes that can be used in accordance with various embodiments of the present invention.

FIG. 5 is a diagram illustrating example procedures for implementing the Gene Network Reconstruction Pipeline (GeneRep) described below in connection with FIG. 4.

FIG. 6 provides a flowchart illustrating operations and processes that can be used in accordance with various embodiments of the present invention.

FIG. 7 is a diagram providing a high-level illustration of an example network Systems Calculation of Optimal Ranking Engine (nSCORE) procedure, described below in connection with FIG. 6.

FIG. 8 provides a flow diagram describing operations for utilizing both the GeneRep and nSCORE procedures to predict and validate biomarkers for treatment of cancers, in accordance with some example embodiments contemplated herein.

FIG. 9 provides a flowchart illustrating operations and processes that can be used in accordance with various embodiments of the present invention for utilizing both the GeneRep and nSCORE procedures to identify and validate master regulators and biomarkers.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

### I. COMPUTER PROGRAM PRODUCTS, METHODS, AND COMPUTING ENTITIES

Embodiments of the present invention may be implemented in various ways, including as computer program products that comprise articles of manufacture. A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### II. EXEMPLARY SYSTEM ARCHITECTURE

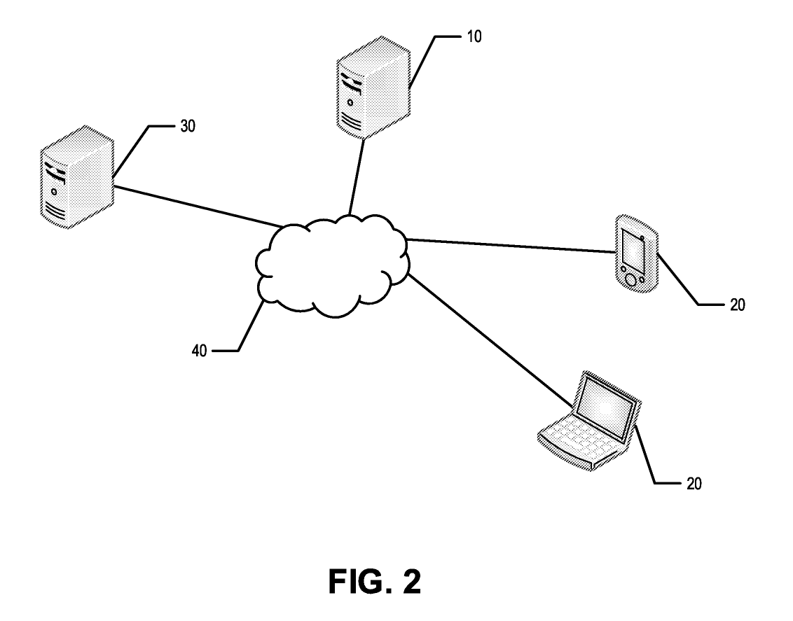

FIG. 2 provides an illustration of an exemplary embodiment of the present invention. As shown in FIG. 2, this particular embodiment may include one or more analysis computing entities 10, one or more user computing entities 20, one or more information/data hosting entities 30, one or more networks 40, and/or the like. Each of these components, entities, devices, systems, and similar words used herein interchangeably may be in direct or indirect communication with, for example, one another over the same or different wired or wireless networks. Additionally, while FIG. 2 illustrates the various system entities as separate, standalone entities, the various embodiments are not limited to this particular architecture.

### 1. Exemplary Analysis Computing Entity

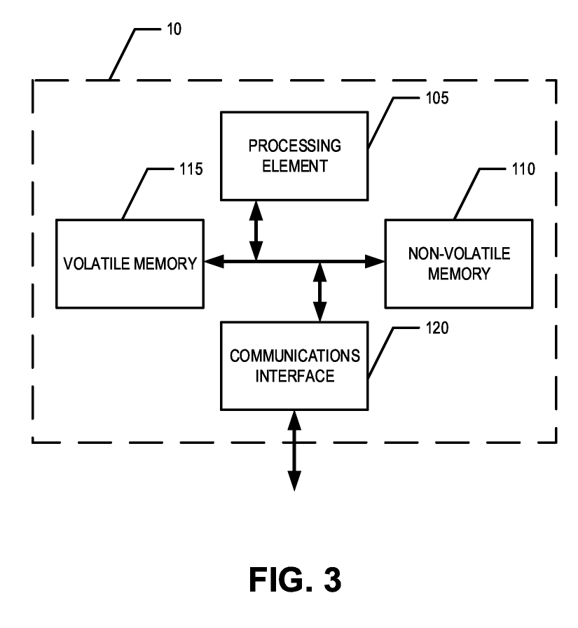

FIG. 3 provides a schematic of an analysis computing entity 10 according to one embodiment of the present invention. In example embodiments, an analysis computing entity 10 may be configured to determine, calculate, compute, estimate, and/or the otherwise determine where to set a threshold cutoff level to maximize sensitivity of a gene expression profile while minimizing the false discovery rate (FDR). In other example embodiments, an analysis computing entity 10 may be configured to discover targetable master regulators within a large gene network requires the ability to rank various genes (or nodes) in the network. In yet further example embodiments, an analysis computing entity 10 may be configured to harvest meaningful data from any type of network and node statistics inputs, even those not related specifically to gene expression profiles. And in additional example embodiments, an analysis computing entity 10 may be used for identifying and validating master regulators and biomarkers.

In general, the terms computing entity, computer, entity, device, system, and/or similar words used herein interchangeably may refer to, for example, one or more computers, computing entities, desktops, mobile phones, tablets, phablets, notebooks, laptops, distributed systems, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

In one embodiment, the analysis computing entity 10 may also include one or more communications interfaces 120 for communicating with various other computing entities, such as by communicating data, content, information, and/or similar terms used herein interchangeably that can be transmitted, received, operated on, processed, displayed, stored, and/or the like.

As shown in FIG. 3, in one embodiment, the analysis computing entity 10 may include or be in communication with one or more processing elements 105 (also referred to as processors, processing circuitry, and/or similar terms used herein interchangeably) that communicate with other elements within the analysis computing entity 10 via a bus, for example. As will be understood, the processing element 105 may be embodied in a number of different ways. For example, the processing element 105 may be embodied as one or more complex programmable logic devices (CPLDs), microprocessors, multi-core processors, co-processing entities, application-specific instruction-set processors (ASIPs), microcontrollers, and/or controllers. Further, the processing element 105 may be embodied as one or more other processing devices or circuitry. The term circuitry may refer to an entirely hardware embodiment or a combination of hardware and computer program products. Thus, the processing element 105 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 105 may be configured for a particular use or configured to execute instructions stored in volatile or non-volatile media or otherwise accessible to the processing element 105. As such, whether configured by hardware or computer program products, or by a combination thereof, the processing element 105 may be capable of performing steps or operations according to embodiments of the present invention when configured accordingly.

In one embodiment, the analysis computing entity 10 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 110, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the analysis computing entity 10 may further include or be in communication with volatile media (also referred to as volatile storage, memory, memory storage, memory circuitry and/or similar terms used herein interchangeably). In one embodiment, the volatile storage or memory may also include one or more volatile storage or memory media 115, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 105. Thus, the databases, database instances, database management systems, data, applications, programs, program modules, scripts, source code, object code, byte code, compiled code, interpreted code, machine code, executable instructions, and/or the like may be used to control certain aspects of the operation of the analysis computing entity 10 with the assistance of the processing element 105 and operating system.

As indicated, in one embodiment, the analysis computing entity 10 may also include one or more communications interfaces 120 for communicating with various other computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the analysis computing entity 10 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra wideband (UWB), infrared (IR) protocols, near field communication (NFC) protocols, Wibree, Bluetooth protocols, wireless universal serial bus (USB) protocols, and/or any other wireless protocol.

Although not shown in FIG. 3, the analysis computing entity 10 may also comprise a user interface (that can include a display coupled to a processing element). For example, the user interface may include or be in communication with one or more input elements, such as a keyboard input, a mouse input, a touch screen/display input, motion input, movement input, audio input, pointing device input, joystick input, keypad input, and/or the like. The analysis computing entity 10 may also include or be in communication with one or more output elements (not shown), such as audio output, video output, screen/display output, motion output, movement output, and/or the like. These input and output elements may include software components such as a user application, browser, graphical user interface, and/or the like to facilitate interactions with and/or cause display of information/data from the analysis computing entity 10, as described herein. The user input interface can comprise any of a number of devices or interfaces allowing the user computing entity 20 to receive data, such as a keypad (hard or soft), a touch display, voice/speech or motion interfaces, or other input device. In embodiments including a keypad, the keypad can include (or cause display of) the conventional numeric (0-9) and related keys (#, *), and other keys used for operating the user computing entity 20 and may include a full set of alphabetic keys or set of keys that may be activated to provide a full set of alphanumeric keys.

As will be appreciated, one or more of the components of the analysis computing entity may be located remotely from other components of the analysis computing entity 10, such as in a distributed system. Furthermore, one or more of these components may be combined with additional components to perform various functions described herein, and these additional components may also be included in the analysis computing entity 10. Thus, the analysis computing entity 10 can be adapted to accommodate a variety of needs and circumstances. As will be recognized, these architectures and descriptions are provided for exemplary purposes only and are not limiting to the various embodiments.

### 2. Exemplary User Computing Entity

In various embodiments, a user computing entity 20 may be configured to exchange and/or store information/data with the analysis computing entity 10. For instance, the user computing entity 20 may be used by a user (e.g., a scientist, lab technician or the like) to provide instructions to the analysis computing entity 10 for structuring or modifying the analysis to be performed by the analysis computing entity 10. The user computing entity 20 may additionally or alternatively receive, information/data from the analysis computing entity 10 or an information/data hosting entity 30 regarding results produced from the operations performed by the analysis computing entity 10. For example, the user computing entity 20 may be configured to determine one or more data sets to use for creating a gene expression profile or for optimizing the gene expression profile and/or may receive an indication of the gene expression profile produced. As another example, the user computing entity 20 may be used to configure an application of the nSCORE procedure by an analysis computing entity 10 (such as by providing an initial data set regarding a network to the analysis computing entity 10) or may receive an indication of the results of an nSCORE procedure (e.g., a set of nodes from a network that are ranked by calculated influence, or the like).

In one embodiment, the user computing entity 20 may include one or more components that are functionally similar to those of the analysis computing entity 10 described above. For example, in one embodiment, each user computing entity 20 may include one or more processing elements (e.g., CPLDs, microprocessors, multi-core processors, co-processing entities, ASIPs, microcontrollers, and/or controllers), volatile and non-volatile storage or memory, one or more communications interfaces, and/or one or more user interfaces.

### 4. Exemplary Index Information/Data Hosting Entity

In various embodiments, the index information/data computing entity 30 may be configured to receive, store, and/or provide information/data comprising gene expression data sets, relevant statistical information, and/or other information/data that may be requested by any of a variety of computing entities.

In one embodiment, an index information/data computing entity 30 may include one or more components that are functionally similar to those of the analysis computing entity 10, user computing entity 20, and/or the like. For example, in one embodiment, each index information/data computing entity 30 may include one or more processing elements (e.g., CPLDs, microprocessors, multi-core processors, co-processing entities, ASIPs, microcontrollers, and/or controllers), volatile and non-volatile storage or memory, one or more communications interfaces, and/or one or more user interfaces.

### III. EXEMPLARY SYSTEM OPERATION

Example embodiments of the present invention address problems identified in computational biology and network theory, and provide solutions that have applicability in a wide range of fields. As mentioned above, example embodiments enable the determination of where to set a threshold cutoff level to maximize the number of recovered gene relationships (edges in a network) while minimizing the false discovery rate (FDR). Some example embodiments exploit the filtered gene expression profile to discover potential master regulators that may be targetable by various perturbagen-based treatments. Other example embodiments facilitate the harvesting of meaningful data from other type of network and node statistics inputs, even those not related specifically to computational biology or the exploitation of inferred gene expression profiles.

A. GeneRep

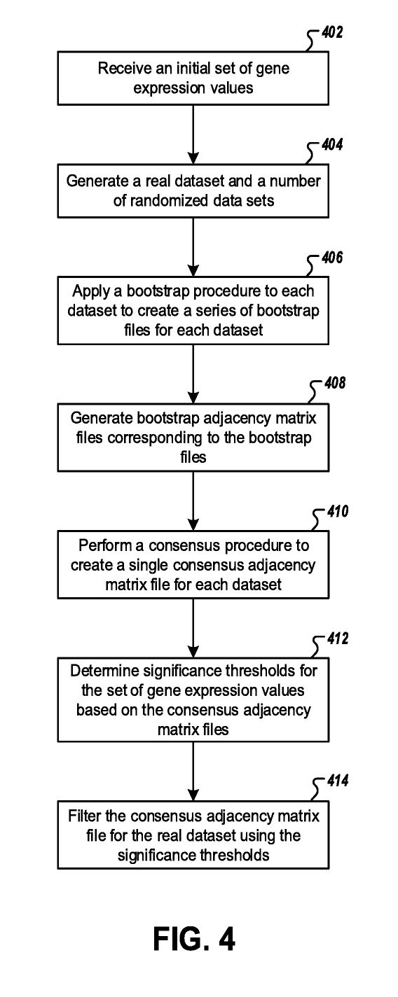

FIG. 4 provides a flowchart illustrating processes and procedures that may be performed in an example embodiment to use GeneRep to filter a gene expression profile based on the calculation of significance thresholds that maximizes sensitivity while minimizing the false discovery rate (FDR). It will be understood that the GeneRep pipeline described herein may be implemented in a variety of ways. In one example, the pipeline utilizes a Python tool called APPLE (ARACNE Processing Pipeline Extensions), which completely automates the analytical steps required to generate a highly reliable regulatory network from gene expression data. APPLE is a command-line tool providing the following nine commands: random, bootstrap, consensus, filter, histogram, extract, convert, stats, and translate, which are described below.

Starting at block 402, an example analysis computing entity 20 receives an initial set of gene expression values. In some embodiments, this dataset may be received from a user computing entity 20, information/data hosting entity 30, or from an external computing entity via network 40.

At block 404 the analysis computing entity 10 generates a real dataset and a number of randomized datasets from the initial set of gene expression values. In an example using the APPLE tool, invocation of the random command generates a number of randomized datasets by shuffling the expression values within each row.

At block 406, the analysis computing entity 10 applies a bootstrap procedure to the real dataset and the randomized datasets to create a series of bootstrap files corresponding to the datasets. In an example using the APPLE tool, the datasets (the single real one and the shuffled ones) undergo a bootstrap procedure implemented by the bootstrap command.

It should be understood that in some embodiments, the number of bootstrap rounds to use and the sample size are specified by a user. In such embodiments, the analysis computing entity 10 may receive the user specification directly via a user interface of the analysis computing entity 10, from a separate user computing entity 20, from stored specifications provided by an information/data hosting entity 30, or via an external computing entity via network 40. Moreover, the series of bootstrap files may be created in the bootstrap procedure are created based on the number of bootstrap rounds and the sample size.

At block 408, the analysis computing entity 10 generates bootstrap adjacency matrix (ADJ) files corresponding to the bootstrap files. In some embodiments, each adjacency matrix file includes an entry (or line) for each hub-gene contained in the corresponding bootstrap file. In this regard, the entry for each hub-gene identifies corresponding edges, wherein each edge comprises a connection for the hub-gene along with mutual information corresponding to the connection. It should be understood that the analysis computing entity 10 may complete this operation using a reverse engineering tool, such as ARACNe (Algorithm for the Reconstruction of Accurate Cellular Networks), which is designed to scale up to the complexity of mammalian cells. ARACNe applies a theoretical information approach to infer gene networks using gene expression data by calculating Mutual Information (MI).

At block 410, the analysis computing entity 10 performs a consensus procedure that utilizes each set of bootstrap adjacency matrix files to generate a single consensus adjacency matrix file for the corresponding dataset. In this regard, each consensus adjacency matrix file identifies only a subset of the edges that occur in the set of bootstrap adjacency matrix files. In some embodiments, determining the subset of the edges to identify in the consensus adjacency matrix for a corresponding dataset itself includes a series of sub-steps. For instance, this operation may include calculating, by the analysis computing entity 10, a support level for each edge by determining a number of bootstrap adjacency matrix files corresponding to the particular dataset that support the edge, and calculating, by the analysis computing entity 10, a FPR for each edge from the bootstrap adjacency matrix files corresponding to the particular dataset that support the edge. The analysis computing entity 10 may then select only those edges having a support level and FPR above a predetermined value. The selected edges are then included as edges in the consensus adjacency matrix file for the corresponding dataset. When using the APPLE tool, this operation can be performed using the consensus command, which writes an edge to the combined file if it was observed in more than S bootstrap files, where S is a user-specified minimum value, and on the basis of its FPR.

At block 412, the analysis computing entity 10 determines, based on the generated consensus adjacency matrix files, significance thresholds for the set of gene expression values. In this regard, performing the consensus procedure may in some embodiments include generating, by the analysis computing entity, a counts file and a statistics file for each consensus adjacency matrix. The counts file may identify a support level of each edge in the consensus adjacency matrix (e.g., by recording the number of edges having each support level), while the statistics file records, for each edge in the consensus adjacency matrix, the support level of the edge, the FPR of the edge, and a sum of the mutual information of the edge, as taken from the bootstrap adjacency matrix files. In such embodiments, the significance thresholds for the set of gene expression values are based on the counts file and the statistics file.

At block 414, the analysis computing entity 10 filters the consensus adjacency matrix file for the real dataset using the determined significance thresholds to produce a final gene expression network stripped of low-significance edges. Filtration thus ensures that edges that do not have sufficient support level or have too large a FPR are not included in the final gene expression network. For instance, using the APPLE tool, the histogram command can be used to produce a histogram of all the MI values in an ADJ file. By comparing the histograms for the real and the shuffled datasets, the analysis computing entity 10 may determine an optimal MI value that maximizes the separation between the real and the shuffled datasets. The analysis computing entity 10 may then use the filter command to take this MI value and generate a new ADJ file containing only the edges with an MI value over this threshold. The process can then be repeated using the sum of the MI values for all edges connected to a hub gene.

It will be understood that a number of stringent filtration methods are contemplated, including 1) removing genes with no expression in >50% of the samples; 2) as a negative control for FPR, creating three non-connected expression profiles by reshuffling the expression values for each gene; 3) for each expression profile, creating a large number (e.g., 100) of bootstraps sets and building a consensus network was built for each, filtered for edges below a support cut-off value; and 4) filtering networks twice more using MI cut-off and sum of MI for each gene cut-off. Moreover, in some embodiments, cut-off values may be chosen to maximize differences in the number of edges retained between the un-shuffled network and the average of three reshuffled networks. In such embodiments, the FPR may be calculated as the ratio of average number of edges in the three negative control networks over the original.

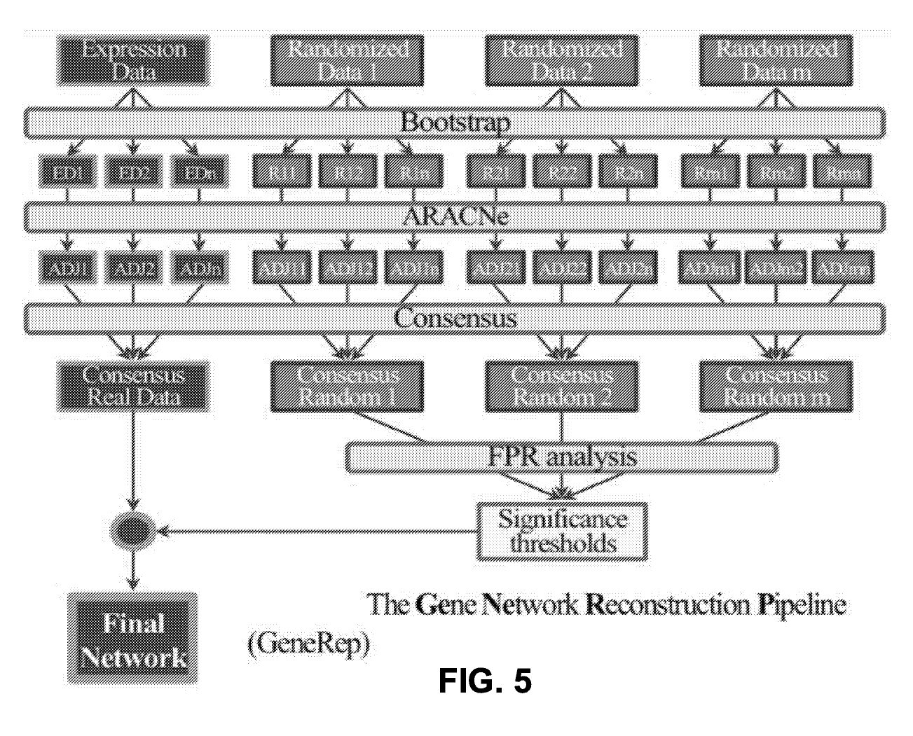

A pictorial illustration of the above procedure is shown in FIG. 5. As shown in FIG. 5, the analysis computing entity 10 may start from a gene-level or transcript-level expression dataset, and utilize the GeneRep pipeline to generate m randomized datasets. From each dataset (real or randomized), n new datasets are generated through a bootstrap procedure. ARACNe is applied to the bootstrap datasets to generate ADJ files, which are then combined into a single consensus network for each dataset. The distributions of edge support and mutual information in the randomized datasets are used to determine significance thresholds, which are then applied to the real consensus network to filter low-significance edges producing a final network.

By performing the operations described above in connection with FIGS. 4 and 5 and the operations illustrated therein, example embodiments are configured to generate a highly reliable regulatory network from gene expression data that maximizes sensitivity while minimizing false discovery rate (FDR) of the network.

It will be understood that the APPLE tool also includes additional commands not mentioned above. These remaining commands provide utilities to print general statistics on one or more ADJ files (stats), extract the edges for a specified set of genes, translate gene identifiers (e.g., from Ensembl to NCBI identifiers), and convert ADJ files to different formats for visualization, including Cytoscape format.

But even generating a filtered regulatory network leaves a significant volume of genes and connections, even if the lowest value connections have been removed. For instance, from about 20,000 genes, there are only limited sets of targets that are important for cancer development. There are several algorithms to identify those targets based on network theory. Genes in a gene regulatory network can be considered as nodes, and the edge between nodes represent relationship between genes. Centrality measures used in graph theory and network analysis such as degree, betweenness, Google PageRank, and eigenvalue can be applied to quantify the influence of a node in a network. The simplest centrality -degree measures the number of direct neighbors. In cell networks, -degree centrality identifies hub genes that interact with many other genes and are important in cell biology.

Betweenness measures the number of shortest paths from all nodes to all others that pass through the node of interest. Google PageRank and eigenvector are based on the concept that the most important node is the one that connects to the most number of important nodes, which can be calculated iteratively or algebraically. In iterative calculation the output score is used as the input for successive calculation until a convergence is assumed. A similar approach is adopted in nSCORE. Various centralities can be used as inputs in nSCORE.

Of note, centralities identify hubs in a static, undisturbed network. Therefore it is highly probable that an anti-cancer drug that targets these nodes will also cause toxicity to normal cells. As a result, it is critical to identify genes that when targeted, only cancer-specific stem-like cells are affected. One approach is to apply centralities to differentially expressed subnetworks extracted from the global network, where nodes in subnetworks are selected from the top of differentially expressed genes (e.g. by FDR) or if their FDR is <a threshold of 0.05. Another approach is to measure a level of change in neighboring nodes surrounding the source node, as successfully used in the ranking algorithms CellNet and Mogrify to identify a set of transcription factors that enhances cell fate conversion, and in the VIPER algorithm. Some of the limitations of current methods are 1) the exclusive use of networks of known relationship; 2) only direct targets allowed; 3) all available node information not fully leveraged; and 4) iterative scoring not implemented. Iterative scoring allows better capturing of network-wide information.

B. nSCORE

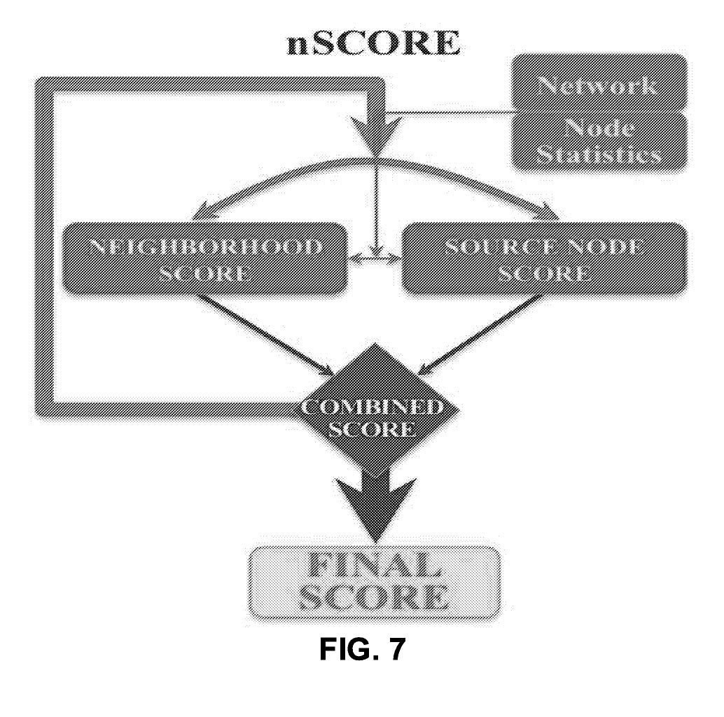

The nSCORE procedure described herein addresses these limitations of current methods for gene target identification. nSCORE comprises a generalized automated node importance scoring framework that incorporates limitless scoring schemes using a set of parameters (FIG. 7). nSCORE combines many existing parameters known individually to influence network properties, and thus can apply to any type of network and node statistics input. The node importance score (niscore) is the aggregation of source node and neighborhood scores. The score is calculated iteratively with the output of the previous calculation serving as the input for the next and so on. Inputs include network (e.g., GeneRep, STRING, or NDEXbio) and node statistics (e.g., log FC, FDR, pvalue, LR or centralities) (Table 1).

TABLE 1
12 input parameters for nSCORE

| Short | | | |
|---|---|---|---|
| name | Long name | Description of Parameter | Examples |
| -s | -step | The number of steps from the source node to neighborhood nodes | |
| -g | -top_gene_statistics | The node statistics used in sub-networks | logFC, pvalue, FDR, LR |
| -l | -gene_statistics_list | The list of gene statistics used to calculate master score | logFC, pvalue, FDR, LR, |
| | | | centralities |
| -r | -nround | The number of rounds in recursive calculation; the score from | |
| | | previous run serves as the input for the next run | |
| -e | -edge_weight = | The edge weight statistics | MI, Rho (normalized MI 0 to |
| | EDGE_WEIGHT | | 1), and unw (unweighted) |
| -p | -edge_weight_exponential | The edge weight exponential used in calculation | square of edge weight |
| -o | -step_exponential | The step exponential used in combination of step scores | |
| -z | -normalize_step_score | To normalize or not to normalize step score for each step (TRUE or | |
| | | FALSE) | |
| -t | -top_genes_proportion | The proportion of top genes from subnetworks of differentially | |
| | | expressed genes from whole network | |
| -k | -use_rank | To use rank instead of gene differential expression value (LR, fdr, | |
| | | logFC) value as inputs | |
| -d | -source_node_inclusion | To include or not to include source node statistics in the calculation | n (no), s(sum) or p |
| | | | (product), m: source node |
| | | | statistics only |
| -a | -average_as_summary_stat | To use average instead of sum of neighbors' scores as the | |
| | | aggregate of neighborhood score | |

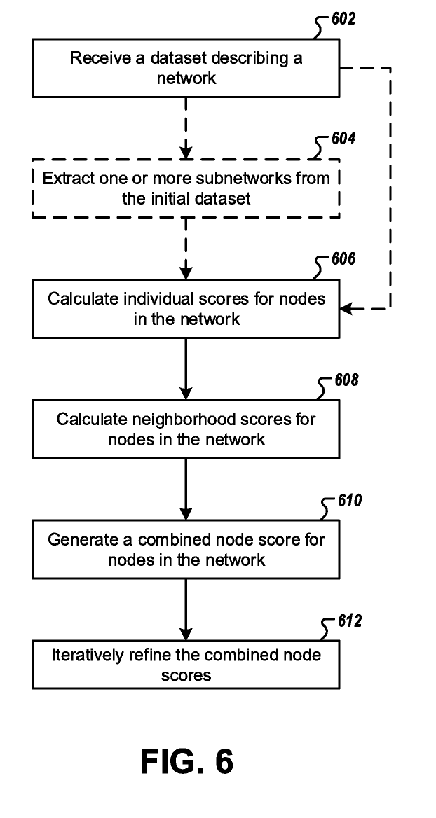

FIG. 6 provides a flowchart illustrating processes and procedures that may be performed by the analysis computing entity 10 in another example embodiment to calculate importance scores for nodes in a network using nSCORE. These processes and procedures may be utilized to identify potential master regulators from a gene expression profile that may in some embodiments be filtered by application of GeneRep as described above. In other embodiments, however, these processes and procedures may be applied to other datasets and contexts as a mechanism for ranking the influence of the nodes describing other types of networks. FIG. 7 shows a diagram providing a high-level illustration of an example of the node influence scoring concept described below in connection with FIG. 6. The nSCORE scoring concept requires as input a dataset describing a network, and a set of node statistics for that dataset. Specific operations implementing the nSCORE framework are illustrated below as performed by an example analysis computing entity 10.

Starting at block 602, the analysis computing entity 10 receives an initial dataset describing a network. As noted previously, this dataset may be received as the result of using GeneRep to filter a gene expression profile network, although in other embodiments, this dataset may regard different types of networks unrelated to computation biology. Having received the initial dataset describing the network, the procedure may advance to either optional block 604 or to block 606 below.

At optional block 604, the analysis computing entity 10 extracts subnetworks from the initial dataset. In some embodiments, these subnetworks may be extracted using the “-top_gene_proportion” and “-g” parameters shown in Table 1. It will be understood that block 604 is optional, and that in some embodiments, the procedure moves directly from block 602 to block 606. The extraction and subsequent use of subnetworks (instead of the whole network) makes the resulting calculations performed in blocks 606 and thereafter faster, although in some instances at the cost of decreased accuracy. And the corollary of this fact is that failure to extract and then use subnetworks produces results having greater accuracy, but at the expense of requiring greater processing resources and/or time.

At block 606, the analysis computing entity 10 calculates individual scores for each node in the one or more subnetworks. However, in embodiments where operation 604 does not occur, the analysis computing entity 10 will instead calculate individual scores for each node in the entire network. If the parameter “-k” of Table 1=TRUE, then the input node statistics are converted to rank value. Either way, the individual score (indscore) for a node i in the network may then be calculated using the formula:

indscoreᵢ=Πₖ₌₁ⁱstatᵢ^{k},indscoreᵢ=statᵢ,

where statᵢ^{k} is a k-th statistic selected from a list of gene statistics included in parameter “-l” of Table 1 above.

Then at block 608, the analysis computing entity 10 calculates neighborhood scores for each node in the one or more subnetworks. However, in embodiments where operation 604 does not occur, the analysis computing entity 10 will instead calculate neighborhood scores for each node in the entire network. In some embodiments, the neighborhood score (nbhscore) for a node i in the network is calculated using the formula:

$${nbhscore}_{i} = {\sum }_{s = 1}^{step} ( \frac{1}{{s}^{o}} * {\sum }_{j = 1}^{{n}_{s}} ( {w}_{i , j}^{p} * {indscore}_{j} ) ) ,$$

where step represents a number of steps from node i to neighborhood nodes

$$\frac{1}{{s}^{o}}$$

comprises a weight penalty based on how far a given node s is from node i; w_{i,j}^{p} is a weight of closeness between nodes i and j; and nₛ is a number of neighbors of node i that require s steps to reach node i. Alternatively, if parameter “-a” from Table 1 above is TRUE, then the neighborhood score (nbhscore) for a node i in the network may instead be calculated using the formula:

$${nbhscore}_{i} = {\sum }_{s = 1}^{step} ( \frac{1}{{s}^{o}} * ( \frac{1}{{n}_{s}} ) * {\sum }_{j = 1}^{{n}_{s}} ( {w}_{i , j}^{p} * {indscore}_{j} ) ) ,$$

where step represents a number of steps from node i to neighborhood nodes;

$$\frac{1}{{s}^{o}}$$

comprises a weight penalty based on how far a given node s is from node i; w_{i,j}^{p} is a weight of closeness between nodes i and j; and nₛ is a number of neighbors of node i that require s steps to reach node i.

At block 610, the analysis computing entity 10 generates a combined node score for each node in the one or more subnetworks. As with the operations performed at blocks 606 and 608, however, in embodiments where operation 604 does not occur, the analysis computing entity 10 will instead calculate combined node scores for each node in the entire network. This combined node score may be generated in a variety of ways, depending on various input parameters identified in Table 1. If parameter “-d” in Table 1 above is =“n”, then this operation comprises setting the combined node score equal to the neighborhood score for the node. If parameter “-d”=“m”, however, this operation comprises setting the combined node score equal to the individual score for the node. If parameter “-d”=“s”, then this operation includes setting the combined node score equal to the neighborhood score for the node plus the product of individual score for the node multiplied the number of nodes in the neighborhood of node i. Finally, if parameter “-d”=“p”, then this operation includes setting the combined node score equal to the individual score for the node multiplied by the neighborhood score for the node.

Finally, at block 612, the analysis computing entity 10 may iteratively refine the combined node scores of each node. Iterative refinement may include repeating the steps performed at blocks 604, 606, 608, and 610 a predetermined number of time or until convergence is reached. And in embodiments where the operations described in connection with block 604 are not performed, the analysis computing entity 10 may instead iteratively refine the combined node scores by repeating only the steps performed at blocks 606, 608, and 610. Convergence may be reached based on user input regarding a desired sum of node-level differences in ranking between consecutive iterations.

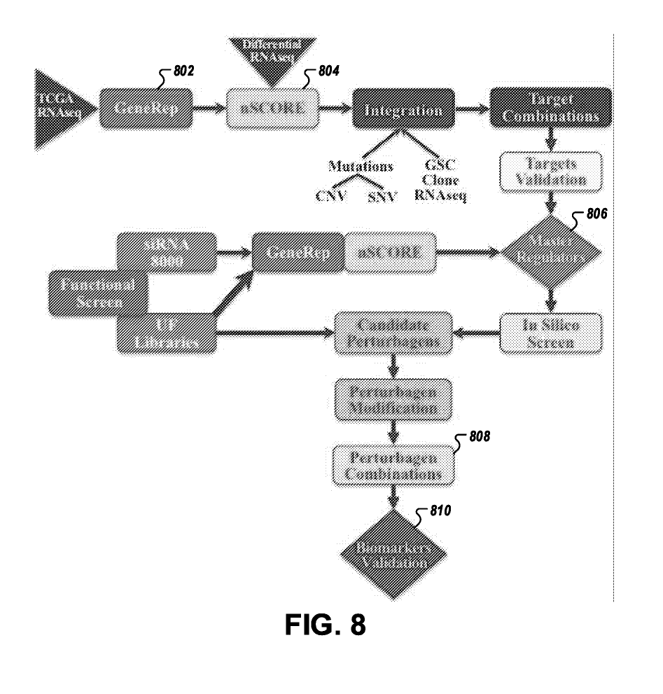

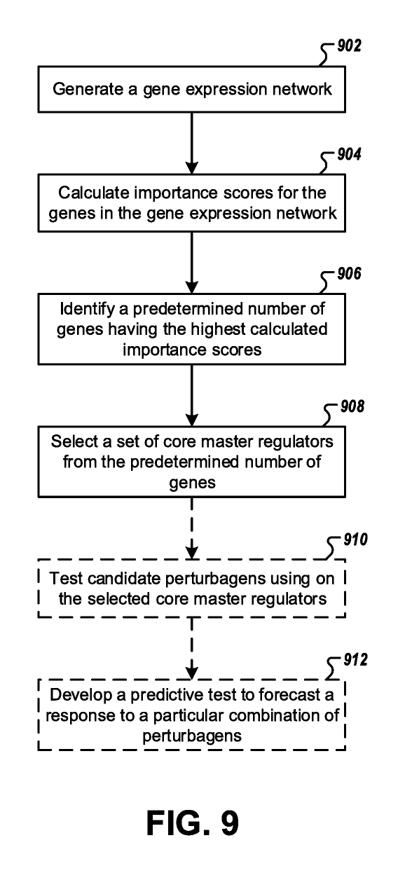

The operations and procedures illustrated in FIGS. 4-7 have many practical applications, although one particular use of these procedures is illustrated in connection with FIGS. 8 and 9 and described in greater detail below.

FIG. 8 shows a diagram providing a high-level illustration of an example of the procedure for targeting master regulators, and will be described below in connection with FIG. 9, in which specific operations are illustrated below as performed by an example analysis computing entity 10.

Starting at block 902 (and in connection with item 802), the analysis computing entity 10 generates a gene expression network using the GeneRep pipeline, as described above in connection with FIG. 4.

At block 904 (and in connection with item 804), the analysis computing entity 10 calculates importance scores for the genes in the gene expression network. In some embodiments, calculating the importance scores for the genes is performed using the nSCORE procedure described above in connection with FIG. 6. It will be understood that this operation can further be optimized in some embodiments. For instance, a standard strategy for machine learning model development may be used to find the best parameter set for a model and estimate its accuracy. For nSCORE, this can be done using publicly available gene expression profiles datasets and a supervised learning k-fold cross-validation approach to assess the predictive performance of the parameter sets. The best performing parameter set may then be evaluated with testing datasets that are not used in the training phase.

First, to generate cell type specific gene regulatory networks, the analysis computing entity 10 uses GeneRep to analyze available gene expression profiles. Then differential expression datasets are collected to train nSCORE. For each given data set, the analysis computing entity 10 may randomly divide the datasets into 2 parts: Part 1 will be for cross-validation and contain 70% of the original, and Part 2 (30%) for testing. Moreover, by randomly dividing Part 1 into equally sized sub-samples, the bulk of the sub-samples can be used as inputs to nSCORE to screen and find the best parameter set (from ˜4000 parameter sets), while the remaining subsample are be retained as validation cases for the round of nSCORE training. The average ranking of all true positives across datasets (master_genes_rank_average or mgra) of the best parameter set for each round of validation serves as the performance criteria for each round of cross-validation. This may then be repeated some predetermined number of times or until all subsamples serve as the validation set one time, at which point the mean of mgra (M-mgra) will be assessed. The 10 fold cross-validation may be run 20 times, each time with a different partition of cross-validation cases. The mean and variance of M-mgra (MM-mgra) can then be estimated. If the MM-mgra is <5 (i.e., true positives are in the top 10 ranked), cross-validation will be considered complete and all samples in the cross-validation cohort (part 1) will be entered as inputs to nSCORE to identify the best parameter set. If MM-mgra is ≥5, a redesign is necessarily of the parameter sets to include more options for each parameter until MM-mgra is <5.

The best parameter set can be validated using the remaining un-used the Part 2 data. After the niscore is reported, a p-value and FDR may be calculated for each gene. The NULL model may then be generated by sample permutations through shuffling of the experimental and control groups.

At block 906, the analysis computing entity 10 identifies a predetermined number of genes having the highest calculated importance scores. The top 20-ranked list will likely contain several master regulators, although a predefined cut off may be set to identify the top 50 ranked niscores, which can be further pared-down by additional operations to maximize the likelihood of capturing true master regulators that the nSCORE algorithm may otherwise miss in a top 20 list.

At block 908 (and in connection with item 806), the analysis computing entity 10 selects a set of core master regulators based on the predetermined number of genes having the highest calculated importance scores. While in some embodiments, this may simply amount to selecting the highest-ranked set of core master regulator candidates, in other embodiments, the nSCORE outputs may be modified with subclonal analysis to more precisely identify relevant common master regulators, and in still further other embodiments, this process may additionally utilize cancer-specific mutational data that enables the operation to potentially identify additional master regulators that otherwise may not even have been identified using the nSCORE algorithm.

At optional block 910 (and in connection with item 808), the analysis computing entity 10 may test candidate perturbagens to identify a best combination of perturbagens based on the selected set of core master regulators. In some examples, this includes determining whether combinations of perturbagens have synergistic or additive effects on reducing neurosphere number and size compared to the single treated or vehicle treated controls. And in some such embodiments, the process may be repeated for all possible combinations of perturbagens, such that the best combination with the largest synergism and the most reduction in neurosphere number/size will be selected.

And finally, at optional block 912 (and in connection with item 808), the analysis computing entity 10 may develop a predictive test to forecast a response to the best combination of perturbagens. The inventors have expected that patient-derived cancerous stem-like cells that have master regulators genes and their local neighborhood genes will be sensitive to the best perturbagen combination targeting these master regulators. Accordingly, the master regulators of responders that perturbagen combinations designed to target should have high niscores in nSCORE. The RNAseq profile of each sample is used to derive the gene differential expression statistics using the EdgeR package (a Bioconductor package for differential expression analysis of digital gene expression data). This gene differential expression statistics table and the GBM network from operation 902 are used as inputs in nSCORE and the niscore for the master regulators of interest will be calculated. The target combination score (TCS) will be defined as: TCS Σᵢ₌₁ⁿ niscoreᵢ, where niscoreᵢ is the niscore of the master regulator i in target combinations.

Cancerous stem-like cells are treated with the best perturbagen combination (identified in operation 910) in a neurosphere formation assay, whose efficacy is assessed as a reduction of number of neurospheres: E=number of neurospheres in treated with vehicle only sample (control)/number of neurospheres in treated sample (experimental). The perturbagen combination is considered successful if E>2, meaning it can reduce the number of neurospheres in half. The receiver operating characteristic (ROC) may be drawn and the area under the curve (AUC-ROC) may be calculated to assess the usefulness of TCS as the response predictor. The cut-off value for TCS may be set at 90% specificity. Samples that have TCS higher than the cut-off value but are not responders (i.e., false positive samples) can then be submitted to RNAseq after treatment, together with the same number of True Positive samples to help in identification of mechanism(s) of resistance. For instance, if the AUC-ROC is low (close to 0.5), most likely one or more perturbagens in the combination may have off-target effects or may interact with each other in an unexpected way. In this case, further study of each perturbagen individually and in combination may determine the mechanism of perturbagen interactions.

As one example proving efficacy of the methodology described in connection with FIGS. 8 and 9, operations described herein were applied by the inventors to identify the core master regulators described in U.S. Provisional Patent Application No. 62/506,413, filed May 15, 2017, the entire contents of which are incorporated herein by reference.

### IV. CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for an analysis computing entity to utilize a computational pipeline to generate a highly reliable regulatory network from gene expression data, the method comprising:
receiving, by the analysis computing entity, an initial set of gene expression values;
generating, by the analysis computing entity, a real dataset and a number of randomized datasets from the initial set of gene expression values;
applying, by the analysis computing entity, a bootstrap procedure to the real dataset and the randomized datasets to create a series of bootstrap files corresponding to the datasets;
for each of the datasets, processing, by the analysis computing entity, the corresponding series of bootstrap files to generate a set of bootstrap adjacency matrix files, wherein each adjacency matrix file includes an entry for each hub-gene contained in the corresponding bootstrap file, wherein the entry for each hub-gene identifies corresponding edges, wherein each edge comprises a connection for the hub-gene along with mutual information corresponding to the connection;
performing, by the analysis computing entity, a consensus procedure that utilizes each set of bootstrap adjacency matrix files to generate a single consensus adjacency matrix file for the corresponding dataset, wherein each consensus adjacency matrix file identifies only a subset of the edges that occur in the set of bootstrap adjacency matrix files;
determining, by the analysis computing entity and based on the generated consensus adjacency matrix files, significance thresholds for the set of gene expression values; and
filtering, by the analysis computing entity, the consensus adjacency matrix file for the real dataset using the determined significance thresholds to produce a gene expression network stripped of low-significance edges.

2. The method of claim 1, further comprising:
receiving, by the analysis computing entity, a user-specified number of bootstrap rounds and sample size, wherein the series of bootstrap files created in the bootstrap procedure are created based on the number of bootstrap rounds and the sample size.

3. The method of claim 1, wherein processing the series of bootstrap files to generate the set of bootstrap adjacency matrix files utilizes a reverse engineering tool for reconstruction of cellular networks.

4. The method of claim 1, further comprising determining the subset of the edges that occur in a particular set of bootstrap adjacency matrix files to identify in the consensus adjacency matrix for a corresponding dataset by:
calculating, by the analysis computing entity, a support level for each edge;
calculating, by the analysis computing entity, a false-positive rate (FPR) for each edge; and
selecting, by the analysis computing entity, only those edges having a support level and FPR above a predetermined value.

5. The method of claim 1, wherein performing the consensus procedure further includes:
generating, by the analysis computing entity, a counts file for each consensus adjacency matrix, wherein the counts file identifies a support level of each edge in the consensus adjacency matrix; and
generating, by the analysis computing entity, a statistics file that records, for each edge in the consensus adjacency matrix, the support level of the edge, the FPR of the edge, and a sum of the mutual information of the edge, as taken from the bootstrap adjacency matrix files,
wherein the significance thresholds for the set of gene expression values are based on the counts file and the statistics file.

6. A method for an analysis computing entity to calculate importance scores for nodes in a network, the method comprising:
(a) receiving, by the analysis computing entity, an initial dataset describing a network;
(b) extracting, by the analysis computing entity, one or more subnetworks from the initial dataset;
(c) calculating, by the analysis computing entity, individual scores for each node in the one or more subnetworks;
(d) calculating, by the analysis computing entity, neighborhood scores for each node in the one or more subnetworks;
(e) generating, by the analysis computing entity, a combined node score for each node in the one or more subnetworks; and
(f) iteratively refining, by the analysis computing entity, the combined node scores.

7. The method of claim 6, wherein calculating an individual score (indscore) for a given node i comprises applying the formula:
indscoreᵢ=Πₖ₌₁ⁱstatᵢ^{k},
where statᵢ^{k} is a k-th statistic selected from a list of gene statistics.

8. The method of claim 6, wherein calculating a neighborhood score (nbhscore) for a given node i comprises applying the formula:
$${nbhscore}_{i} = {\sum }_{s = 1}^{step} ( \frac{1}{{s}^{o}} * {\sum }_{j = 1}^{{n}_{s}} ( {w}_{i , j}^{p} * {indscore}_{j} ) ) ,$$
where step represents a number of steps from node i to neighborhood noaes;
$$\frac{1}{{s}^{o}}$$
comprises a weight penalty based on how far a given node s is from node i; w_{i,j}^{p} is a weight of closeness between nodes i and j; and nₛ is a number of neighbors of node i that require s steps to reach node i.

9. The method of claim 6, wherein calculating a neighborhood score (nbhscore) for a given node i comprises applying the formula:
$${nbhscore}_{i} = {\sum }_{s = 1}^{step} ( \frac{1}{{s}^{o}} * ( \frac{1}{{n}_{s}} ) * {\sum }_{j = 1}^{{n}_{s}} ( {w}_{i , j}^{p} * {indscore}_{j} ) ) ,$$
where step represents a number of steps from node i to neighborhood nodes;
$$\frac{1}{{s}^{o}}$$
comprises a weight penalty based on how far a given node s is from node i; w_{i,j}^{p} is a weight of closeness between nodes i and j; and nₛ is a number of neighbors of node i that require s steps to reach node i.

10. The method of claim 6, wherein generating the combined node score for a given node includes one of:
setting the combined node score equal to the individual score for the node;
setting the combined node score equal to the neighborhood score for the node;
setting the combined node score equal to the neighborhood score for the node plus a product produced by multiplying the individual score for the node by a value comprising a number of other nodes in the neighborhood of the node; or
setting the combined node score equal to the individual score for the node multiplied by the neighborhood score for the node.

11. The method of claim 6, wherein iteratively refining the combined node scores includes:
repeating steps (b), (c), (d), and (e) a predetermined number of time or until convergence is reached.

12. A method for identifying and validating one or more master regulators and biomarkers, the method comprising:
generating, by an analysis computing entity, a gene expression network;
calculating, by the analysis computing entity, importance scores for the genes in the gene expression network;
identifying a predetermined number of genes having the highest calculated importance scores;
selecting a set of core master regulators based on the predetermined number of genes having the highest calculated importance scores;
testing candidate perturbagens to identify a best combination of perturbagens based on the selected set of core master regulators; and
developing a predictive test to forecast a response to the best combination of perturbagens.

13. An apparatus comprising at least one processor and at least one memory storing computer program code, the at least one memory and the computer program code configured to, with the processor, cause the apparatus to at least:
receive an initial set of gene expression values;
generate a real dataset and a number of randomized datasets from the initial set of gene expression values;
apply a bootstrap procedure to the real dataset and the randomized datasets to create a series of bootstrap files corresponding to the datasets;
for each of the datasets, process the corresponding series of bootstrap files to generate a set of bootstrap adjacency matrix files, wherein each adjacency matrix file includes an entry for each hub-gene contained in the corresponding bootstrap file, wherein the entry for each hub-gene identifies corresponding edges, wherein each edge comprises a connection for the hub-gene along with mutual information corresponding to the connection;
perform a consensus procedure that utilizes each set of bootstrap adjacency matrix files to generate a single consensus adjacency matrix file for the corresponding dataset, wherein each consensus adjacency matrix file identifies only a subset of the edges that occur in the set of bootstrap adjacency matrix files;
determine, based on the generated consensus adjacency matrix files, significance thresholds for the set of gene expression values; and
filter the consensus adjacency matrix file for the real dataset using the determined significance thresholds to produce a gene expression network stripped of low-significance edges.

14. A computer program product comprising at least one non-transitory computer-readable storage medium having computer-executable program code instructions stored therein, the computer-executable program code instructions comprising program code instructions that, when executed, cause a computer to at least:
receive an initial set of gene expression values;
generate a real dataset and a number of randomized datasets from the initial set of gene expression values;
apply a bootstrap procedure to the real dataset and the randomized datasets to create a series of bootstrap files corresponding to the datasets;
for each of the datasets, process the corresponding series of bootstrap files to generate a set of bootstrap adjacency matrix files, wherein each adjacency matrix file includes an entry for each hub-gene contained in the corresponding bootstrap file, wherein the entry for each hub-gene identifies corresponding edges, wherein each edge comprises a connection for the hub-gene along with mutual information corresponding to the connection;
perform a consensus procedure that utilizes each set of bootstrap adjacency matrix files to generate a single consensus adjacency matrix file for the corresponding dataset, wherein each consensus adjacency matrix file identifies only a subset of the edges that occur in the set of bootstrap adjacency matrix files;
determine, based on the generated consensus adjacency matrix files, significance thresholds for the set of gene expression values; and
filter the consensus adjacency matrix file for the real dataset using the determined significance thresholds to produce a gene expression network stripped of low-significance edges.

15. The apparatus of claim 13, wherein the at least one memory and the computer program code are further configured to, with the processor, cause the apparatus to at least receive a user-specified number of bootstrap rounds and sample size, wherein the series of bootstrap files created in the bootstrap procedure are created based on the number of bootstrap rounds and the sample size.

16. The apparatus of claim 13, wherein processing the series of bootstrap files to generate the set of bootstrap adjacency matrix files utilizes a reverse engineering tool for reconstruction of cellular networks.

17. The apparatus of claim 13, wherein the at least one memory and the computer program code are further configured to, with the processor, cause the apparatus to at least determine the subset of the edges that occur in a particular set of bootstrap adjacency matrix files to identify in the consensus adjacency matrix for a corresponding dataset by:
calculating a support level for each edge;
calculating a false-positive rate (FPR) for each edge; and
selecting only those edges having a support level and FPR above a predetermined value.

18. The computer program product of claim 14, wherein the computer-executable program code instructions further comprise program code instructions that, when executed, cause the computer to at least receive a user-specified number of bootstrap rounds and sample size, wherein the series of bootstrap files created in the bootstrap procedure are created based on the number of bootstrap rounds and the sample size.

19. The computer program product of claim 14, wherein processing the series of bootstrap files to generate the set of bootstrap adjacency matrix files utilizes a reverse engineering tool for reconstruction of cellular networks.

20. The computer program product of claim 14, wherein the computer-executable program code instructions further comprise program code instructions that, when executed, cause the computer to at least determine the subset of the edges that occur in a particular set of bootstrap adjacency matrix files to identify in the consensus adjacency matrix for a corresponding dataset by:
calculating a support level for each edge;
calculating a false-positive rate (FPR) for each edge; and
selecting only those edges having a support level and FPR above a predetermined value.
